(19) United States
(12) Patent Application Publication
(10) Pub. No.: US 2013/0245634 A1
(43) Pub. Date: Sep. 19, 2013

(54) PLUNGER SYSTEM FOR INTRAOCULAR LENS SURGERY

(71) Applicant: Kyle Brown, Fort Worth, TX (US)

(72) Inventor: Kyle Brown, Fort Worth, TX (US)

(21) Appl. No.: 13/618,318

(22) Filed: Sep. 14, 2012

Related U.S. Application Data
(60) Provisional application No. 61/579,887, filed on Dec. 23, 2011.

(51) Int. Cl. A61F 2/16

(52) U.S. Cl. CPC A61F 2/1667
USPC 606/107

(57) ABSTRACT

Various systems, apparatuses, and processes may be used for intraocular lens surgery. In particular implementations, a system for intraocular lens surgery may include a body and a plunger. The body may, for example, include an outer wall and an inner wall, where the inner wall defines a passage through the body and includes a first guide member. The plunger may, for example, be adapted to move within the passage and include a first end, a second end, and a second guide member. The first end may be adapted to be engaged by a user for moving the plunger within the passage, and the second end may include a tip adapted to interface with an intraocular lens and having an asymmetric cross-section. The second guide member may interact with the first guide member to rotate the tip when the plunger is moved through the body.

### RELATED APPLICATIONS

This application claims priority to U.S. provisional application Ser. No. 61/579,887, filed on Dec. 23, 2011, the contents which are incorporated herein by reference.

### BACKGROUND

The present invention relates to optical surgery, and more specifically to surgical replacement of a patient's lens.

The human eye, in simple terms, functions to provide vision by transmitting and refracting light through a clear outer portion called the cornea and focusing the image by way of the lens onto the retina at the back of the eye. The quality of the focused image depends on many factors including the size, shape, and length of the eye, and the shape and transparency of the cornea and lens.

When trauma, age, or disease causes the lens to become less transparent, vision deteriorates because of a reduction in light transmitted to the retina. This deficiency in the eye's lens is medically known as a cataract. The treatment for this condition is often surgical removal of the lens and implantation of an artificial lens, often termed an intraocular lens (IOL).

An IOL is often foldable and inserted into the eye through a relatively small incision by being advanced through an IOL insertion cartridge, which causes the IOL to fold. The IOL is typically advanced through the insertion cartridge by a plunger-like device.

### BRIEF SUMMARY

In one general implementation, a system for intraocular lens (IOL) surgery may include a body and a plunger. The body may include an outer wall and an inner wall, with the inner wall defining a passage through the body and including a first guide member. The plunger may be adapted to move within the passage and include a first end, a second end, and a second guide member. The first end may be adapted to be engaged by a user for moving the plunger within the passage, and the second end may include a tip adapted to interface with an intraocular lens and having an asymmetric (e.g., rectangular) cross-section. The second guide member may interact with the first guide member to rotate the tip when the plunger is moved through the body. The rotation of the tip may, for example, be approximately 90 degrees as the plunger is advanced relative to the body.

In certain implementations, the first guide member may be a protuberance that extends into the passage, and the second guide member may be a channel adapted to receive the protuberance. In some implementations, the first guide member may be a ramped surface, and the second guide member may be a protuberance that extends into the passage and is adapted to following the ramped surface.

Various implementations may include one or more features. For example, by having a rotating plunger tip, a system for intraocular lens surgery may allow a plunger to have adequate height at initial contact with the IOL, which can facilitate lens folding, while having reduced height when arriving at the insertion point, which can facilitate using smaller incisions. As another example, the rotation function may occur and be controlled automatically, with no end user action required. Thus, repeatable results may be obtained.

The details and features of various implementations will be conveyed by the following description, along with the drawings.

### BRIEF DESCRIPTION OF THE SEVERAL VIEWS OF THE DRAWINGS

FIGS. 1A-B show an example plunger system for intraocular lens surgery.

FIG. 2 is a cross-sectional view of an example IOL interface of an example plunger.

FIG. 3 is a partial cross-sectional view of an end of an example plunger having a rotatable annular ring.

FIG. 4 shows a cross-sectional view of another example plunger system.

FIGS. 5A and 5B are partial detail views of another example plunger guide system.

FIGS. 6 and 7 show front and side views of an example plunger.

FIG. 8A and 8B are perspective views of an example intraocular lens insertion cartridge.

FIGS. 9A and 9B are cross-sectional views of an example plunger guide system.

FIG. 10 is a flowchart illustrating an example process for intraocular lens surgery.

### DETAILED DESCRIPTION

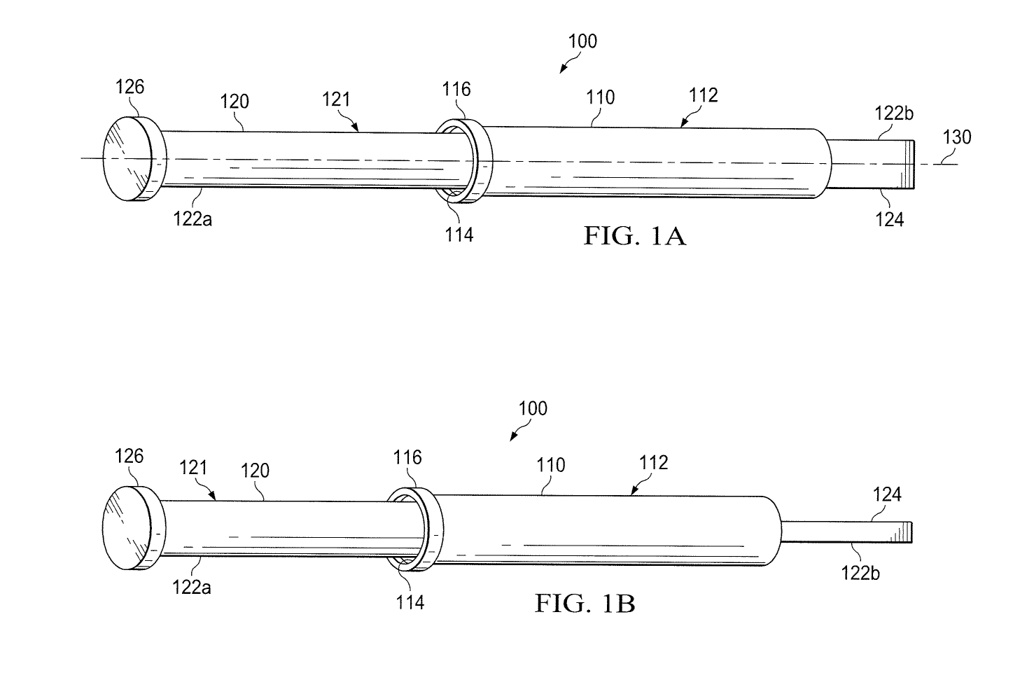

FIGS. 1A and 1B illustrate an example plunger system 100 for intraocular lens (IOL) surgery. Plunger system 100 includes a shell 110 and a plunger 120, which is adapted to move within shell 110.

Shell 110 includes a body 112 that has a passage 114 therethrough. As illustrated, body 112 is generally cylindrical in shape, and so is passage 114. Shell 110 also includes an annular ring 116 that extends from body 112. Annular ring 116 may be sized to allow a user, such as a physician or other medical professional, to manually grasp the system 100 (e.g., with a pair of fingers). Shell 110 may be made of plastic, metal, or any other appropriate material.

Plunger 120 includes a body 121 and has a first end 122a and a second end 122b. As illustrated, first end 122a is generally cylindrical and sized to fit inside passage 114 while still allowing plunger 120 to move relative thereto. Second end 122b is opposite first end 122a and includes an IOL interface 124. As shown in the illustrated example, IOL interface 124 may have a rectangular cross-section. IOL interface 124 may have other cross-sectional shapes in other implementations. For example, in some instances, the IOL interface 124 may have an oval or elliptical cross-section. However, the IOL interface 124 may have other suitable cross-sections. Also, in some implementations, IOL interface 124 may be approximately 2-3 mm in width. Further, in some implementations, the IOL interface 124 may be integrally formed on the body 121. In other implementations, IOL interface 124 may not be an integral with the body 121. For example, IOL interface 124 may be a separate component that is coupled to the body 121.

IOL interface 124 is operable to interface with an IOL and to advance the IOL through an IOL insertion cartridge. IOL interface 124 may be made of an injection-molded elastomer, polymer (e.g., polypropylene or styrene), metal, or any other appropriate material.

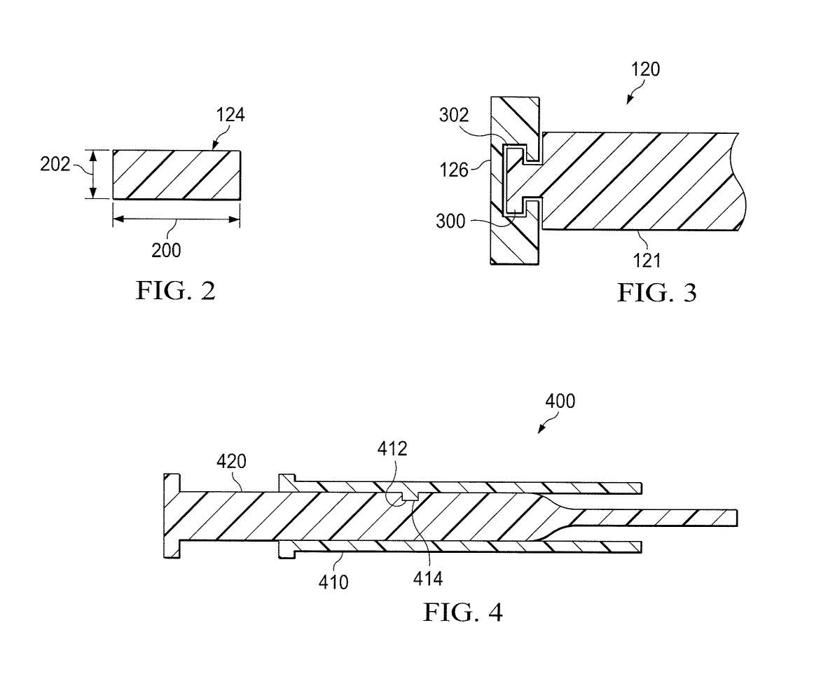

FIG. 2 shows a cross-sectional view of an example IOL interface 124. The illustrated example IOL 124 interface includes a rectangular cross-section having a width 200 and a height 202. In some implementations, one cross-sectional dimension of the IOL interface 124 may be greater than another cross-sectional dimension. For example, in some instances, a first dimension of the IOL interface cross-section may be twenty percent greater in size than a second cross-sectional dimension. Thus, again referring to FIG. 2, in some instances, the width 200 may be twenty percent greater than the height 202. In some instances, the size of one cross-sectional dimension may be more than twenty percent larger than the second cross-sectional dimension. For example, in some instances, the size of one cross-sectional dimension may be twice the size of another cross-sectional dimension. Thus, in some instances, the width 200 may be up to two times the size of the height 202. Thus, it is within the scope of the present disclosure that a size of a first cross-sectional dimension of an IOL interface, such as IOL interface 124, may be within the range of twenty percent larger to two times the size of a second cross-sectional dimension of the IOL interface. In other instances, one cross-sectional dimension may be greater than two times the size of a second cross-sectional dimension. Further, although the description is made with reference to an example IOL interface having the rectangular cross-sectional shape shown in FIG. 2, the scope is not so limited. Rather, the relative sizes of the cross-sectional dimensions also apply to IOL interfaces having other cross-sectional shapes.

First end 122a may generally taper to the shape of IOL interface 124, or there may be a distinct transition from the shape of first end 122a to the shape of IOL interface 124. Thus, the manner in which the first end 122a transitions into the shape of IOL interface 124 may be in any suitable manner. Plunger 120 may be made of plastic, metal, or any other appropriate material.

Plunger 120 may also include an annular ring 126 that extends from body 121 at end 122a. Annular ring 126 may assist a user in manipulating plunger 120 to advance it through shell 110. In some instances, the annular ring 126 may be rotatably attached to the body 121. For example, as shown in FIG. 3, a protrusion 300 formed on the plunger body 121 is received into a recess 302 formed in the annular ring 126. Thus, the annular ring 126 is retained on and freely rotatable relative to the plunger body 121. In other instances, a protrusion formed on the annular ring 126 may be received into a recess or opening formed in the body 121 so that the annular ring 126 is rotatable relative to the body 121. However, other ways of rotatably coupling the annular ring 126 to the body 121 may be used and are within the scope of the disclosure.

Rotatably coupling the annular ring 126 and body 121 is advantageous because, a user may utilize a finger, such as a thumb, to apply pressure to the plunger 120 during use. As the plunger 120 moves relative to the shell 110, the plunger 120 rotates relative thereto, as discussed in more detail below. If the annular ring 126 is rotatable relative to the body 121, the user's thumb does not move relative to the annular ring 126 as the plunger body 121 rotates relative to the shell 110. This improves control of the plunger 120 and, hence, the plunger system 100, during use.

Shell 110 and plunger 120 may include guide members that rotate plunger 120 relative to shell 110 as plunger 120 moves therethrough. For example, the guide members may be a protrusion received into a groove. FIG. 4 shows a cross-sectional view of an example plunger system 400. In the illustrated example, shell 410 includes a protrusion 412 received into a groove 414 of plunger 420. Thus, in some implementations, a protrusion maybe on the shell, such as shell 110 or 410, while the groove may be in the plunger, such as plunger 120 or 420. In other implementations, the protrusion may form part of the plunger and be received into a groove formed in the shell

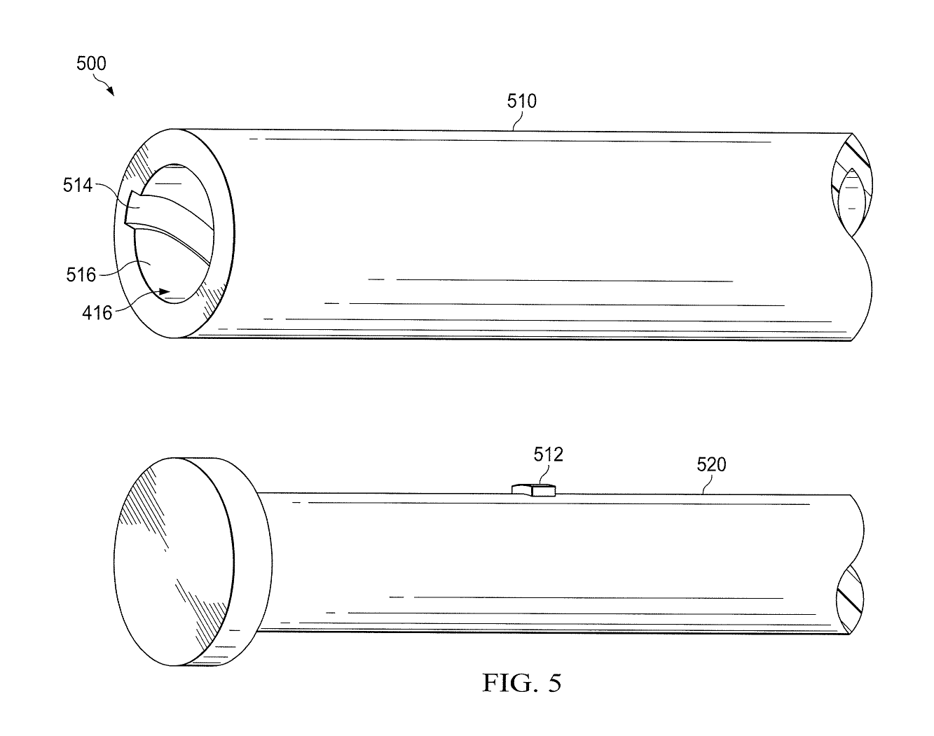

FIG. 5 shows a partial detail view of example plunger system 500 in a disassembled configuration. As shown, plunger 520 includes a protrusion 512 extending from an exterior surface 513 of the plunger 520. Shell 510 includes a groove 514 formed in an inner wall 516 of the shell 510. The groove 514 is adapted to receive the protrusion 512. The shell 510 also defines a passage 518 adapted to receive the plunger 520. Similar to the examples described above in which the protrusion forms part of the shell and the groove is formed in the plunger, the protrusion 512 and groove 514 cooperatively interact to rotate the plunger 520 as the plunger 520 is displaced within the shell 510.

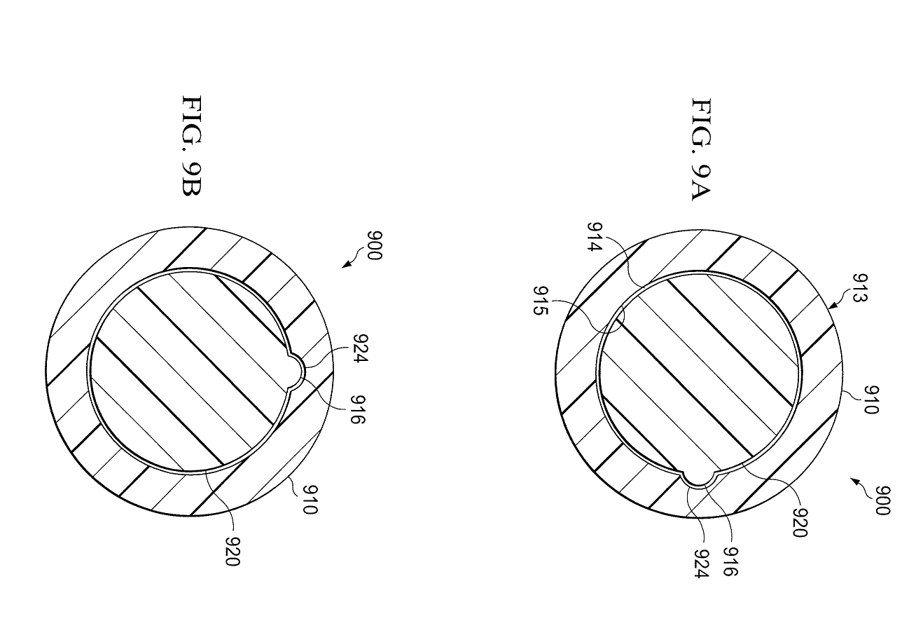

In operation, before plunger 120 is moved through passage 114, IOL interface 124 is in a first orientation, as depicted in FIG. 1A. This orientation may, for example, be beneficial for engaging an IOL, which may be in an IOL insertion cartridge. As plunger 120 is moved through passage 114, the IOL interface 124 advances an IOL through the IOL insertion cartridge, such as IOL insertion cartridge 800 shown in FIGS. 8A and 8B. As the IOL interface 124 reaches a desired location within the IOL insertion cartridge, interaction between the protrusion and the groove (which may be similar to those shown in FIGS. 4, 5, 9A, or 9B, for example) causes the plunger 120 to rotate about its longitudinal axis 130 to a different orientation, as depicted in FIG. 1B. In particular implementations, the difference between the beginning orientation and the ending orientation may be approximately 90 degrees. Plunger 120 may then advance the IOL through the end of the IOL insertion cartridge.

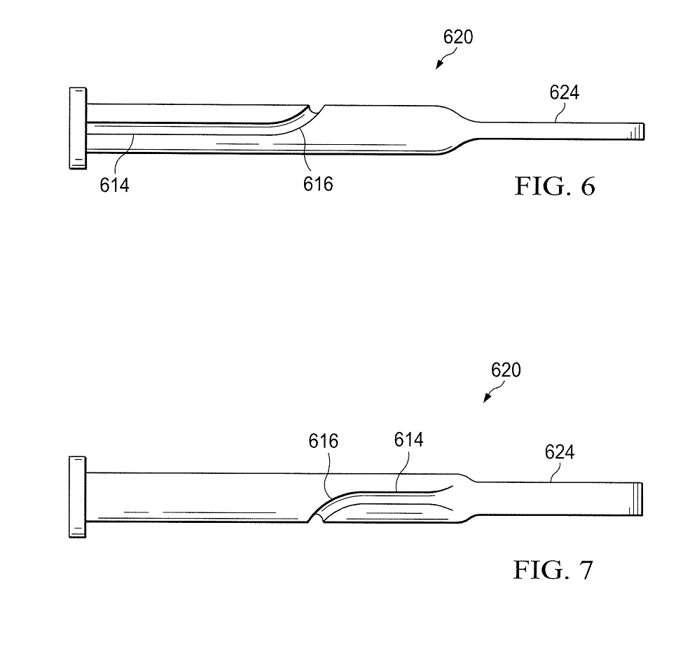

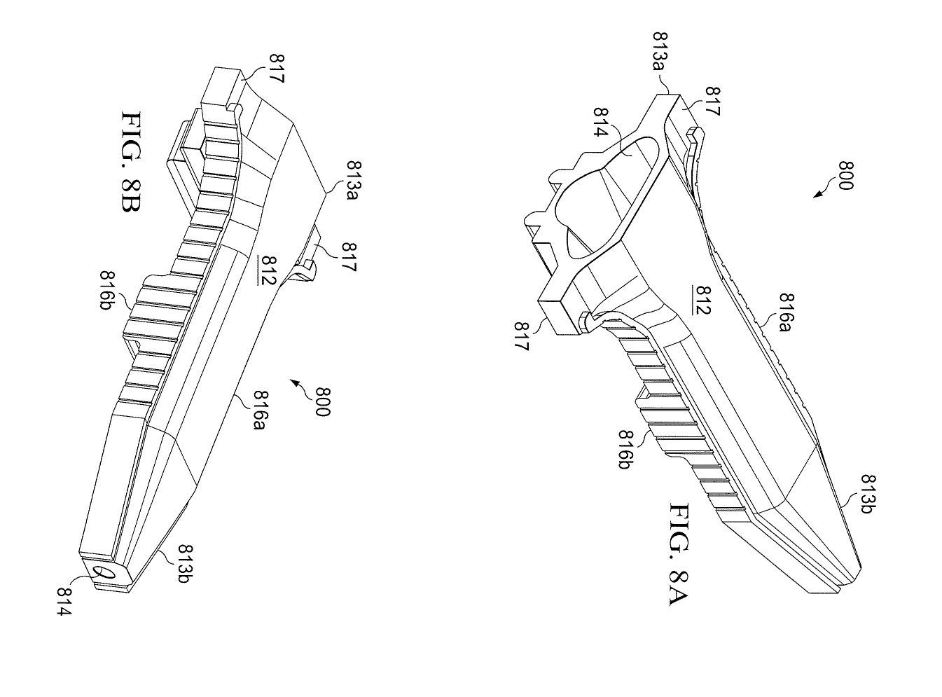

FIGS. 6 and 7 show side and top views, respectively, of an example plunger 620. The plunger 620 includes a groove 614 and an IOL interface 624. In the example shown, the groove 614 includes a curved portion 616. The curved portion 616 is operable to rotate the plunger 620 a desired amount. In the illustrated example, the curved portion 616 is operable to rotate the plunger 620 approximately 90 degrees as it is advanced through a shell as a result of interaction between the groove 614 and a protrusion of the shell received therein. In other instances, the amount of rotation of the plunger relative to the shell as a result of interaction between the groove and protrusion during advancement of the plunger through the shell may be greater or less than 90 degrees.

FIGS. 6 and 7 show that the cross-sectional shape of the groove 614 has a generally rectangular shape. However, in other instances, the groove 614 may have other shapes. For example, the groove 614 may have a rounded shape, a semi-circular shape, a v-shape, a square shape, or any other desired shape.

Further, as illustrated by FIGS. 6 and 7, the curved portion 616 forms a portion of the total length of groove 614. A length of the curved portion 616 relative to a total length of the groove 614 may be selected to be any desired amount. Thus, in some implementations, the curved portion 616 may form only a small portion of the overall length of the groove 614. In such instances, the IOL interface 624 is made to rotate a desired amount over a short distance, resulting in a rapid rotation of the IOL interface 624 for a particular rate of displacement of the plunger 120. In other implementations, the length of the curved portion 616 may form a larger portion of the total length of the groove 614. Consequently, rotation of the IOL interface 624 may be more gradual as the plunger 620 is advanced through the corresponding shell, such as shell 110, 410, or 510, for example, at the particular rate of displacement. As such, not only may the amount by which the IOL interface is rotated relative to the shell be varied, the amount of rotation of the IOL interface per axial displacement of the plunger within the shell may also be varied.

FIGS. 8A and 8B illustrate an example IOL insertion cartridge 800. IOL insertion cartridge 800 facilitates the insertion of an IOL into a patient's eye. IOL insertion cartridge 800 includes a body 812 that has ends 813a and 813b and a passage 814 through the body. During surgery, a foldable IOL, which may be made of silicone, soft acrylics, hydrogels, or other appropriate materials, is moved through passage 814 in preparation for insertion into the eye. IOL insertion cartridge 800 also includes sides 816a and 816b, which assist in grasping the IOL insertion cartridge. Sides 816a and 816b taper outward to form wings 817, which also assist in grasping the IOL insertion cartridge.

As shown, passage 814 has an asymmetric bore at end 813a, which assists in folding an IOL. A common IOL may be approximately 6 mm in diameter, and with haptics can be up to around 13 mm. However, surgical incisions are typically much smaller (e.g., 2-3 mm in width). An IOL is therefore typically folded before insertion through the incision. Passage 814 also tapers along its length to an elliptical bore at end 813b to assist in folding an IOL. Thus, as an IOL is advanced through passage 814, the IOL is folded due to the shape of the passage. The end of the passage may be the injection point through which the lens is inserted into an eye.

In certain implementations, IOL insertion cartridge 800 may be molded as a single piece from any suitable thermoplastic, such as polypropylene. In particular implementations, the thermoplastic may contain a lubricity enhancing agent.

Although FIGS. 8A, 8B illustrate one example implementation of an IOL insertion cartridge, other implementations may include fewer, additional, and/or a different arrangement of components. In some implementations, for example, body 810 may not include wings 817. Additionally, passage 814 may have a symmetrical bore (e.g., round or elliptical).

System 100 is generally usable with pre-loaded and manually loaded IOL insertion cartridges with substantially oval or elliptically nozzle tip shapes where the nozzle tip height is smaller than the nozzle tip width. Shapes of this type are typical of many delivery systems due to their compatibility with the incision.

System 100 provides a variety of features. For example, system 100 allows a plunger to have adequate height at initial contact with the IOL, which can facilitate lens folding, while having reduced height when arriving at the insertion point, which can facilitate using smaller incisions. This is accomplished by the ability of the plunger, and particularly the IOL interface, to rotate during translation through the IOL insertion cartridge. This rotation provides for improved folding of the IOL as well improved delivery of the IOL through a small nozzle tip.

Typically, with one piece plungers, dimensional compromises have to be made to arrive at a best-fit plunger height that adequately accomplishes the lens folding and delivery tasks and still fits through a small nozzle tip. The disadvantage to this approach is that the plunger is often not tall enough initially to provide functional performance under extreme delivery conditions and not small enough to provide as much clearance in the nozzle tip as is preferred. Very tight tolerances are also required for plungers of this design, which increase initial development and manufacturing costs. Moreover, tight control of manufacturing processes over time is also required in order to maintain the product dimensional specifications. Thus, system 100 provides a one-piece plunger component which reduces initial design and manufacturing complexity. Moreover, the functions of system 100 are designed to occur and be controlled automatically, with no end user action required.

Although FIGS. 1A and 1B illustrate one implementation of a plunger system for IOL surgery, other implementations may include fewer, additional, and/or a different arrangement of components. For example, a plunger system may not include annular ring 116 or annular ring 126. As another example, body 121 may not be a cylinder. For instance, body 121 could have a “plus-shaped” cross-section composed of two intersecting webs. As a further example, IOL interface 124 may not be rectangular in cross section. For example, in some instances, IOL interface 124 may have an elliptical or oval cross-sectional shape. However, the body 121 of the plunger 120 may have any suitable shape such that the plunger 120 is operable to rotate within the shell 110 as described herein.

FIGS. 9A and 9B illustrate another example plunger system 900. As illustrated, system 900 includes a shell 910 and a plunger 920. FIG. 9A illustrates a cross section of shell 910 and plunger 920 at a location of the plunger 920 having a protrusion 916. FIG. 9A represents a cross section at a first point during operation of the system 900. FIG. 9B illustrates a cross section of shell 910 and plunger 920 the location of the plunger 920 having the protrusion 916 at a subsequent point during operation of the system 900.

Shell 910 includes an outer wall 913 and an inner wall 915. Inner wall 913 defines a passage 914. Also, a groove 924 is formed in the inner wall 913. The protrusion 916 is received within the groove 924. While FIGS. 9A, 9B illustrate the groove 924 as formed in the shell 910 and the protrusion 916 formed on the plunger 920, as explained above, this configuration may be switched. That is, the groove may be formed in the plunger, and the protrusion may be formed on the inner surface of the shell. As also explained above, groove 924 may have any shape operable to alter the rotational orientation of plunger 920 as it is moved through shell 910.

In certain modes of operation, groove 924 engages protrusion 916 as plunger 920 is advanced through passage 914, as illustrated in FIG. 9A. As plunger 920 is advanced further along passage 914, interaction between protrusion 916 and groove 924 causes plunger 920 to rotate counterclockwise. However, in other implementations, plunger 920 may be rotated clockwise. As illustrated, protrusion 916 remains engaged with groove 924 to define an orientation of the plunger 920 relative to the shell 910.

In the illustrated example, the plunger 920 is rotated approximately 90 degrees as a result of interaction between the groove 924 and the protrusion 916. However, as explained above, the amount of rotation of the plunger 920 relative to the shell 910 may be selected to be any desired amount. Further, while a cooperating groove and protrusion configuration is illustrated, the scope of the disclosure is not so limited. Rather, any cooperating features operable to rotate the plunger and shell relative to each other as the plunger is axially translated through the shell may be used.

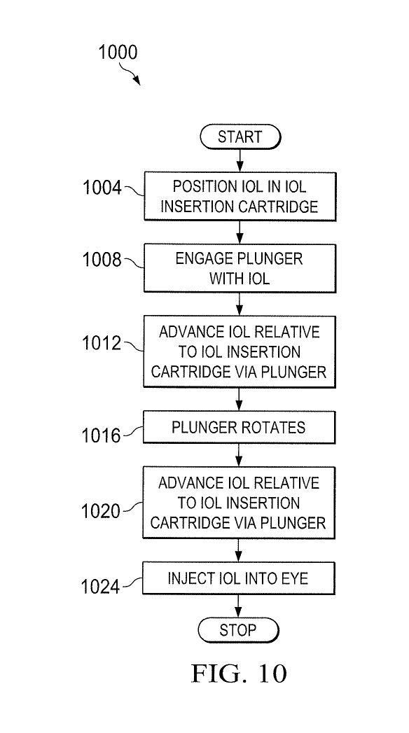

FIG. 10 illustrates an example process 1000 for using a plunger system for intraocular lens surgery. Process 1000 may, for instance, be performed using a plunger system. For example, the process 1000 may be performed using one or more of the example plunger system described herein.

Process 1000 includes positioning an IOL in an IOL insertion cartridge (operation 1004). The IOL insertion cartridge may, for example, be similar to IOL insertion cartridge 800.

Process 1000 also includes engaging a plunger of a plunger system with the IOL (operation 1008). The plunger may, for example, be engaged with the IOL by advancing the tip of the plunger until it touches the IOL.

Process 1000 also includes advancing the IOL relative to the IOL insertion cartridge using the plunger (operation 1012). For example, the plunger may be advanced relative to the shell of the plunger system, which may move the IOL in the IOL insertion cartridge. The IOL may be folded by advancement through the IOL insertion cartridge.

As the IOL is moved relative to the IOL insertion cartridge, the plunger rotates (operation 1016). In particular implementations, the plunger may rotate approximately 90 degrees due a guide system arrangement (e.g., a groove and protrusion arrangement) of the plunger system. However, the plunger may be rotated any desired amount.

Process 1000 also includes further advancing the IOL relative to the IOL insertion cartridge using the plunger (operation 1020). The additional advancement may further fold the IOL.

Additionally, process 1000 includes injecting the IOL into an eye (operation 1024). For example, the IOL may be injected when it reaches the end of the IOL insertion cartridge.

Although process 1000 illustrates one example of a process for using a plunger system for intraocular lens surgery, other processes for using a plunger system for IOL surgery may include fewer, additional, and/or a different arrangement of operations. For example, a process may not include positioning the IOL in the IOL insertion cartridge. The IOL may, for instance, have been pre-positioned in the IOL insertion cartridge. As another example, a process may call for engaging the plunger system with the IOL insertion cartridge.

The various implementations discussed and mentioned herein have been used for illustrative purposes only. The implementations were chosen and described in order to explain the principles of the disclosure and the practical application and to allow those of ordinary skill in the art to understand the disclosure for various implementations with various modifications as are suited to the particular use contemplated. Thus, the actual physical configuration of components may vary. For example, the mentioned size(s) of components and their illustrated sizing relative to each other may vary based on application. Moreover, the shapes of one or more components may vary depending on application. Thus, the illustrative implementations should not be construed as defining the only physical size, shape, and relationship of components.

The terminology used herein is for the purpose of describing particular implementations only and is not intended to be limiting. As used herein, the singular form “a”, “an”, and “the” are intended to include the plural forms as well, unless the context clearly indicates otherwise. It will be further understood that the terms “comprises” and/or “comprising,” when used in the this specification, specify the presence of stated features, integers, steps, operations, elements, and/or components, but do not preclude the presence or addition of one or more other features, integers, steps, operations, elements, components, and/or groups therefore.

The corresponding structure, materials, acts, and equivalents of all means or steps plus function elements in the claims below are intended to include any structure, material, or act for performing the function in combination with other claimed elements as specifically claimed. The description of the present implementations has been presented for purposes of illustration and description, but is not intended to be exhaustive or limited to the implementations in the form disclosed. Many modifications and variations will be apparent to those of ordinary skill in the art without departing from the scope and spirit of the disclosure.

A number of implementations have been described for a plunger system for intraocular lens surgery, and several others have been mentioned or suggested. Moreover, those skilled in the art will readily recognize that a variety of additions, deletions, modifications, and substitutions may be made to these implementations while still providing a plunger system for intraocular lens surgery. Thus, the scope of the protected subject matter should be judged based on the following claims, which may capture one or more concepts of one or more implementations.

1. A system for intraocular lens surgery, the system comprising:
a body comprising an outer wall and an inner wall, the inner wall defining a passage through the body and comprising a first guide member; and
a plunger adapted to move within the passage, the plunger comprising:
a first end comprising a tip adapted to interface with an intraocular lens, the tip comprising an asymmetric cross-section; and
a second guide member, wherein the second guide member interacts with the first guide member to rotate the tip when the plunger is moved through the body.

2. The system of claim 1, wherein the cross-section of the tip is rectangular.

3. The system of claim 1, wherein one cross-section dimension of the tip is greater than another cross-section dimension of the tip by greater than twenty percent.

4. The system of claim 1, wherein:
the first guide member comprises a protuberance that extends into the passage; and
the second guide member comprises a channel adapted to receive the protuberance.

5. The system of claim 1, wherein:
the first guide member comprises a ramped surface; and
the second guide member comprises a protuberance that extends into the passage and is adapted to following the ramped surface.

6. The system of claim 1, wherein the first guide member and the second guide member cooperate to rotate the plunger approximately 90 degrees when the plunger is advanced from a first position relative to the body to a second position relative to the body.

7. The system of claim 1, wherein the plunger further comprises a second end adapted to be engaged by a user for moving the plunger within the passage.

8. A method for intraocular lens surgery, the method comprising:
advancing a plunger of an intraocular lens plunger system that comprises a shell and a plunger through an intraocular lens insertion cartridge; and
allowing the plunger to rotate relative to the shell due to a guide system in the plunger system.

9. The method of claim 8, wherein the guide system comprises a first guide member on the shell and a second guide member on the plunger.

10. The method of claim 9, wherein:
the first guide member comprises a protuberance extending into a passage of the shell; and
the second guide member comprises a channel in the plunger that is adapted to receive the protuberance.

11. The method of claim 8, further comprising engaging the plunger with an intraocular lens.

12. The method of claim 11, further comprising injecting the intraocular lens into an eye.

13. The method or claim 8, wherein the rotation is approximately 90 degrees when the plunger is advanced from a first position relative to the body to a second position relative to the body.
